(19) United States
(12) Patent Application Publication
(10) Pub. No.: US 2014/0196724 A1
(43) Pub. Date: Jul. 17, 2014

(54) TRACHEAL DILATION APPARATUS AND METHOD OF MANUFACTURE

(71) Applicant: COVIDIEN LP, Mansfield, MA (US)

(72) Inventor: Sean Morris, Athlone (IR)

(73) Assignee: COVIDIEN LP, Mansfield, MA (US)

(21) Appl. No.: 13/742,146

(22) Filed: Jan. 15, 2013

(51) Int. Cl. A61M 16/04

(52) U.S. Cl. CPC A61M 16/0472
USPC 128/207.29; 29/592

(57) ABSTRACT

A tracheal dilator system and method of manufacture are provided, suitable for dilating a passageway into a patient airway. In one embodiment, a medical system having a tracheal dilator is provided. The tracheal dilator includes a first plurality of openings and a guide lumen configured to provide a pathway for the insertion of a guide wire during use of the tracheal dilator. The tracheal dilator further includes a first flow lumen configured to fluidly couple the first plurality of openings to a medical device, wherein the medical device is configured to provide a suctioning force through the first plurality of openings during use of the tracheal dilator.

### BACKGROUND

The present disclosure relates to a tracheal dilation techniques, and more particularly to a tracheal dilation via a dilation cannula structure.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide range of applications exist for artificial ventilation, which may call for the use of tubes that are inserted into a patient. Such tubes may include endotracheal tubes, tracheostomy tubes, and so forth. In the latter case, the tubes are typically inserted into an opening or stoma formed in the neck and trachea of the patient. In both cases, the tubes may be used for artificial ventilation or for assisting patient ventilation. The stoma is typically formed either surgically, through a procedure such as a cricothyroidotomy, tracheostomy, or through a micro-surgical procedure such as percutaneous dilation. Cricothyroidotomy requires the use of a surgical team working in a sterilized environment to create an opening in the cricothyroid membrane, thus providing access to the patient's airway. The procedure typically involves the cauterizing of blood vessels, and typically has the patient undergoing general anesthesia.

Percutaneous dilation entails using an instrument, such as a needle or a scalpel, to make a small opening between the tracheal rings on a frontal or anterior region of the patient's neck. The needle or scalpel may then be inserted through the opening in the tracheal rings to allow a passageway into the patient's airway. A dilator may then be pushed inwardly towards the trachea to enlarge the stoma. It would be beneficial to provide for a more efficient dilation apparatus.

### SUMMARY

The present disclosure provides a novel dilation device that includes one or more lumens with openings fluidly coupled to a pumping device. In a first mode of operation, the pumping device, such as a pump or a ventilator, may be used to provide positive airflow suitable for expelling a gas (e.g., air) through the dilator openings and creating a cushion effect. By “lubricating” the stoma opening, the cushion effect may aid in the insertion of the dilator, thus minimizing tearing of tissue and additionally minimizing the clinician's insertion effort. In a second mode of operation, the pumping device may provide for a suctioning force suitable for vacuuming fluids (e.g., secretions) and tissue particles during the dilation, thus minimizing bleeding and patient discomfort. Accordingly, any bleeding may be minimized and effluent entering the airway may be eliminated.

The positive airflow may be provided at the beginning of insertion of the dilator, and then the pumping device may switch to providing suctioning once the dilator tip is inserted, or vice versa. In other modes of operation, only positive airflow or only suctioning may be used. In certain embodiments, the dilation device may be a curved and/or cone shape dilator, similar to a horn, with increasing diameter from a distal tip to a proximal base. As the dilator penetrates the stoma, the increasing diameter of the dilator may gradually expand the stoma until a desired size is reached, suitable for the insertion of a tracheostomy tube. By using the multiple lumens connected to openings disposed on the dilator for positive airflow and/or suctioning, the dilation techniques described herein may minimize trauma and provide for a more efficient and faster dilation procedure.

In accordance with one embodiment, a medical system having a tracheal dilator is provided. The tracheal dilator includes a first plurality of openings and a guide lumen configured to provide a pathway for the insertion of a guide wire during use of the tracheal dilator. The tracheal dilator further includes a first flow lumen configured to fluidly couple the first plurality of openings to a medical device, wherein the medical device is configured to provide a suctioning force through the first plurality of openings during use of the tracheal dilator.

In a similar arrangement, a tracheal dilator is provided. The tracheal dilator includes a guide cannula configured to provide a pathway for the insertion of a guide wire during use of the tracheal dilator. The tracheal dilator additionally includes a first flow cannula comprising a first plurality of openings, wherein the first flow cannula is configured to fluidly couple the first plurality of openings to a medical device, and wherein the medical device is configured to provide a positive flow of a gas through the first plurality of openings during use of the tracheal dilator.

Also provided is a method for manufacturing a tracheostomy dilator. The method includes manufacturing a guide cannula configured to provide a pathway for the insertion of a guide wire during use of the tracheostomy dilator. The method additionally includes manufacturing a first flow cannula comprising a first plurality of openings, wherein the first flow cannula is configured to fluidly couple the first plurality of openings to a medical device, and wherein the medical device is configured to provide a positive flow of a gas through the first plurality of openings during use of the tracheal dilator.

### BRIEF DESCRIPTION OF THE DRAWINGS

Advantages of the disclosed techniques may become apparent upon reading the following detailed description and upon reference to the drawings in which:

FIG. 1 is a sectional view of a patient's tracheal region and an insertion of a percutaneous needle;

FIG. 2 is a sectional view of a guide wire and the percutaneous needle inserted into the tracheal region of FIG. 1;

FIG. 3 is a sectional view illustrating an embodiment of a multiple lumen dilator disposed in a tracheal region;

FIG. 4 is a sectional view of the of an embodiment of the multiple lumen dilator of FIG. 3, illustrating a guide lumen and a flow lumen;

FIG. 5 is a sectional view of a distal tip of an embodiment of the multiple lumen dilator of FIG. 3;

FIG. 6 is a side view of an embodiment of the multiple lumen dilator of FIG. 3 having a curved shape;

FIG. 7 is a perspective view of an embodiment of a manifold of the multiple lumen dilator of FIG. 6;

FIG. 8 is a rear view of a of an embodiment of the manifold of the multiple lumen dilator of FIG. 7;

FIG. 9 is a view of flow lumen openings having circular shapes;

FIG. 10 is a view of flow lumen openings having teardrop shapes;

FIG. 11 is a view of flow lumen openings having reverse teardrop shapes; and

FIG. 12 is a view of flow lumen openings having slit shapes.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

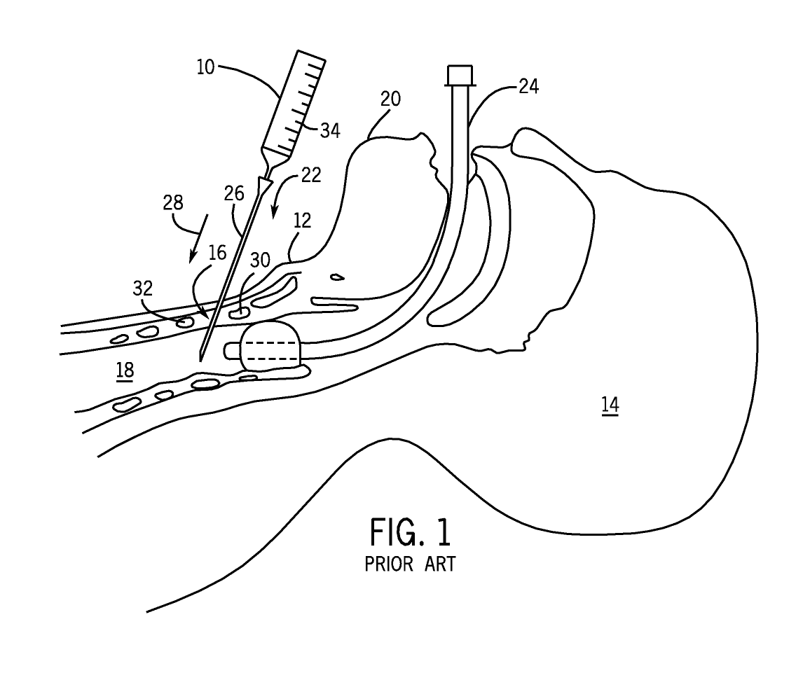

FIG. 1 is a sectional view illustrating a placement of a percutaneous needle 10 in a trachea 12 of a patient 14. By inserting the percutaneous needle 10 into the trachea 12, an initial opening or tracheal passageway 16 into an airway 18 is created, suitable for dilation. As depicted, the patient 14 is disposed in a supine position, with a chin 20 slightly elevated. In certain circumstances, a rostal traction on the tracheal 12 may be applied so as to gain neck hyperextension and better access to a frontal neck region 22. General or local anesthesia may be used to dull or eliminate any discomfort during the dilation procedure. Additionally, the patient 14 may be intubated, such as by using an endotracheal tube 24. Indeed, the systems and methods disclosed herein enable a dilation procedure with artificial respiration kept in situ. It is also to be noted that the systems and methods disclosed herein enable dilation without artificial respiration support (e.g., without the endotracheal tube 24).

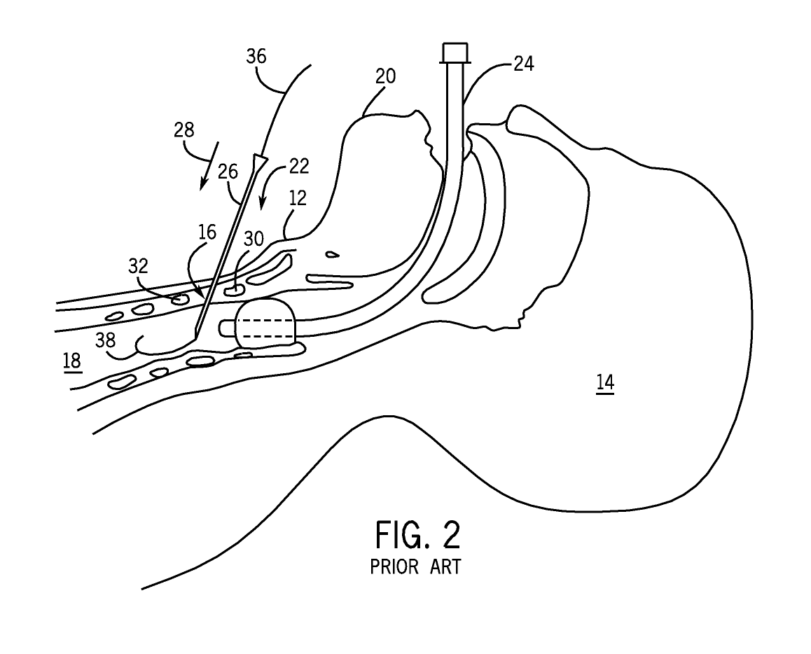

As depicted, a cannula 26 of the percutaneous needle 10 may be inserted in a direction 28, and enter the trachea 12 between a first 30 and a second 32 tracheal rings. As the percutaneous needle 10 is advanced in the direction 28, an aspiration of air through the needle 10 may indicate that the needle 26 has reached a desired position inside of the patient airway 18. Other methods useful in verifying that the cannula 26 is in the desired position may be used, such as a bronchoscopial survey, an ultrasound survey, and the like. It is also to be noted that other instruments may be used in creating the initial passageway 16 through the trachea 12. For example, a scalpel may also be used to provide a vertical or horizontal slit passageway 16 into the trachea 12. By using minimally invasive techniques to breach the trachea 12, scarring and other unsightly neck trauma may be minimized or avoided. Likewise, major bleeding during the dilation procedure may be eliminated. Once a clinician has verified that the needle cannula 26 has reached the desired position inside the airway 18, a body 34 of the needle 10 may be removed. A guide wire, such as a J-tip guide wire, may then be inserted through the cannula 26 of the needle 10, as described in more detail below with respect to FIG. 2.

FIG. 2 is a sectional view depicting the insertion of a J-tip guide wire 36 into the patient's airway 18. Because the figure contains like elements found in FIG. 1, these like elements are denoted using like reference numbers. As illustrated, the guide wire 36 is disposed inside of the needle cannula 26 and inserted so that a generally curved tip 38 is positioned inside the patient's airway 18. Using a guide wire, such as the J-tip guide wire 36, may enable a more efficient insertion of the dilation systems described herein. However, the dilation systems described herein may also be inserted into the trachea 12 without the use of any type of guide wire.

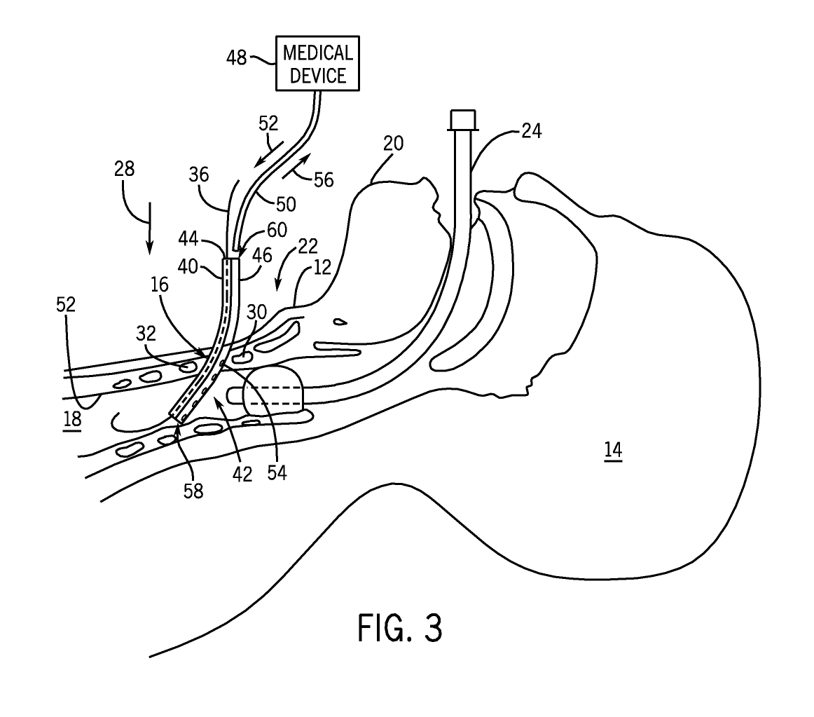

When the J-tip guide wire 36 is used, the curved tip 38 may cause less trauma because the curved portion of the tip 38 is less likely to puncture the patient airway 18. That is, the curved tip 38 may prevent a “poking” or dagger effect. Once the curved tip 38 is inside the airway 18, the clinician may insert the guide wire 36 into a hollow shaft of a dilator, and then “slide” the dilator over the guide wire 36 to position a dilator partially inside of the patient airway 18, as depicted in FIG. 3.

FIG. 3 is a sectional view illustrating a multi-lumen dilator 40 having a distal portion 42 positioned inside of the tracheal passageway 16. As mentioned above, the guide wire 36 may be disposed inside a guide lumen 44 of the multi-lumen dilator 40 and used as a guide into the patient airway 18. One or more flow lumens 46 may also be provided. In one embodiment, the guide lumen 44 may not be used and the single (or multiple) lumens 46 may be used. The flow lumens 46 may be fluidly coupled to a medical device 48, such as a pump (e.g., vacuum pump and/or positive pressure pump) and/or a ventilator, by using one or more conduits 50. In a first operating mode, the medical device 48 may provide for positive airflow suitable for providing a gas (e.g., air, medicines) in the direction 52 inwardly into the dilator lumen(s) 46, thus delivering a positive pressure flow that may exit through a plurality of openings 54. The aforementioned gas flow may then create a cushion of air, which may surround the dilator 40 and provide for gaseous lubrication during the insertion (or retrieval) of the dilator 40. The positive fluid flow pressure may also be controllable, thus providing for the air cushion effect in a wide variety of patient anatomies, including neonatal, pediatric, and adult patients. The medical device may use the ideal gas law, i.e., P×V=n×R×T, where P is a fluid flow pressure suitable for inflating a volume V at a temperature T based on the number of moles n of a gas and on the ideal gas constant R. Accordingly, the desired volumetric pressure may be found. For example, the inflation P may be between about 15 cm H₂O and 10,000 cm H₂O.

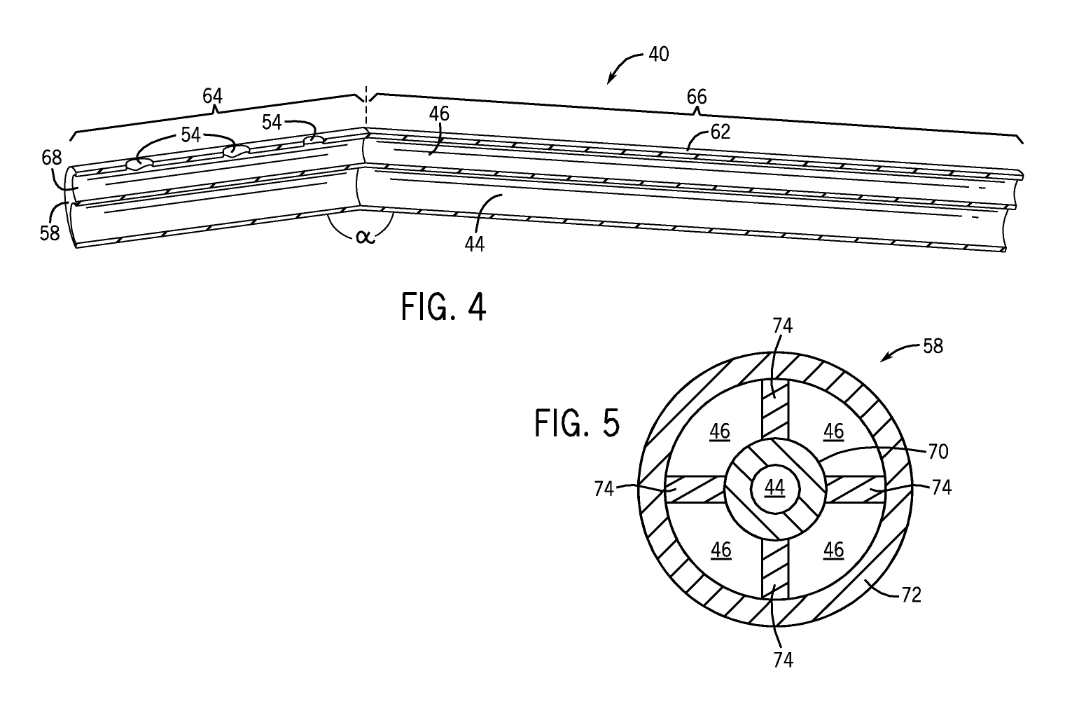

In a second operating mode, the medical device 48 may create a vacuum suitable for suctioning secretions, gas, and loose particles through the openings 54 and into the medical device 48 in a direction 56. The suctioning mode of operations may be particularly useful in certain situations, including emergency response situations such as when the patient is more prone to bleeding. The suctioning force may be varied by the clinician to accommodate various usage scenarios, for example, from heaving fluid flows to light fluid flows. Accordingly, patient secretions entering the airway 18 may be minimized or eliminated. By providing for gas flow and/or the vacuum force, the medical device 48 may enable a more efficient dilation while minimizing trauma. In certain embodiments, the multi-lumen dilator 40 includes an increasing diameter from a distal tip 58 to a proximal base 60, which may be useful in expanding the passageway 16 as the dilator 40 is moved inwardly towards the airway 18. To aid in the insertion into the airway 18, the dilator 40 may include a generally conically-shaped body, as described in more detail with respect to FIG. 4.

Turning now to FIG. 4, the figure is a cross-sectional view of an embodiment of the multi-lumen dilator 40 having a conically-shaped body 62 and an angled portion 64 having an angle α. In the depicted embodiment, the angled portion 64 may include the openings 54, while a portion 66 may not include any openings 54. In another embodiment, the portion 66, or section of the portion 66, may also include the openings 54. In one embodiment, the portion 64 may be between 1/10 to ½ of a total length of the dilator 40. The angle α may enable the use of a levering force useful in providing added leverage when dilating the stoma. For example, the clinician may insert the distal tip 58 into the stoma with only a partial (or complete) section of the portion 64 in the stoma, and then use the tissues in the passageway 16 as a fulcrum to further dilate and insert the dilator 64 further inwardly into the airway 18. The angle α may be between 90° to 270°. Indeed, in one embodiment, the angle a may be 180°, thus forming a straight, unbent line between the portions 64 and 66.

Also depicted are the lumens 44 and 46. As mentioned previously, the lumen 44 may be used as a guide lumen suitable for use of the guide wire 36 as shown in FIG. 3. Once the guide wire 36 is disposed inside of the lumen 44, the dilator 40 may then follow the guide wire 36 inwardly into the airway 18. The lumen 46 may be fluidly coupled to the medical device 48 and used to provide for gaseous flow outwardly from the openings 54, and/or used to suction secretions and particulates inwardly into the lumen 46 and the medical device 48. Accordingly, the multi-lumen dilator 40 may be used to more easily dilate the stoma as well as to minimize or eliminate secretions from entering into the patient airway 18. The dilator 40 may be manufactured from a material such as a polyvinylchloride, a polyurethane, thermoplastic elastomers, a polycarbonate plastic, silicon, an acrylonitrile butadiene styrene (ABS), or a polyvinyl chloride (PVC). The lumens 44 and 46 may be molded, overmolded, computer numerical control (CNC) machined, milled, or otherwise formed into the desired shape (e.g., tubular shape).

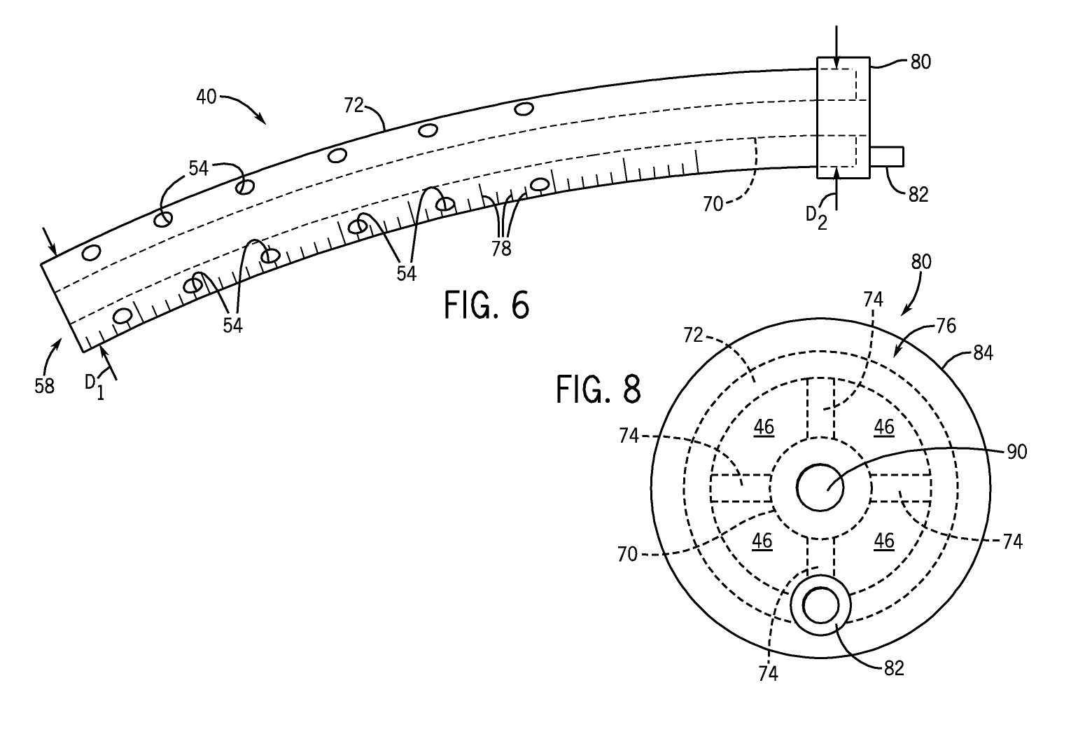

In another embodiment, as shown in FIG. 5, the flow lumens 46 may be circumferentially disposed around the guide lumen 44, thus disposing the plurality of openings 54 circumferentially around the guide lumen 44. More specifically, FIG. 5 is a front view of the distal tip 58 showing four fluid lumens 46 disposed circumferentially around the guide lumen 44. In the depicted embodiment, the guide lumen 44 may be included as a conduit formed by a tubular member or inner cannula 70. The inner cannula 70 may be disposed inside of a tubular member or outer cannula 72, and attached to the outer cannula 72 by using support members 74. The support members 74 may include a length equal to the length of the inner and/or outer cannula 70, 72. The support members 74 may provide a chamber or lumen 46 by dividing a region between exterior surfaces of the inner cannula 70 and inner surfaces of the outer cannula 70. More specifically, each lumen 46 may be formed as a chamber by using, for example, neighboring support members 74 as side walls, the inner cannula's 70 outer surface as a floor, and the outer cannula's 72 inner walls as a roof. The support members 74 may include rectangular members, posts, columns, and other shapes suitable for dividing the region between the exterior surface of the inner cannula 70 and the inner surface of the outer cannula 70 into the lumens 46. The support members 74 may be adhered (glued or thermally bonded) to the cannulae 70 and 72, or may be molded or overmolded to connect the inner cannula 70 to the outer cannula 72. By providing for multiple lumens 46, the openings 54 may be disposed in a variety of locations, including locations circumferentially surrounding the outer cannula 72, as described in more detail below with respect to FIG. 6.

FIG. 6 is a side view of the dilator 40 including a plurality of the openings 54 disposed circumferentially around outer surfaces of the outer cannula 72. Also shown in dashed lines is the inner guide cannula 70 disposed inside of the outer cannula 72. It is to be understood that, while in the depicted embodiment the dilator 40 includes a curved shaped, in other embodiments, the dilator 40 may include a straight shape or an angled shape (e.g., as shown in FIG. 4). As mentioned above, the dilator 40 may increase in diameter from the distal tip 58 to a proximal base 76. For example, the distal tip 58 may include a diameter D1 of between 10 mm to 10 mm, while the proximal base 76 may include a diameter D2 of between 5 mm to 15 mm or more. The dilator 40 may also include markings 78 disposed throughout a portion or throughout the entirety of the length of the outer cannula 72. The markings may include measurements in millimeters (mm), inches, or other units useful in visually indicating a depth of penetration of the dilator 40 into the patient's neck.

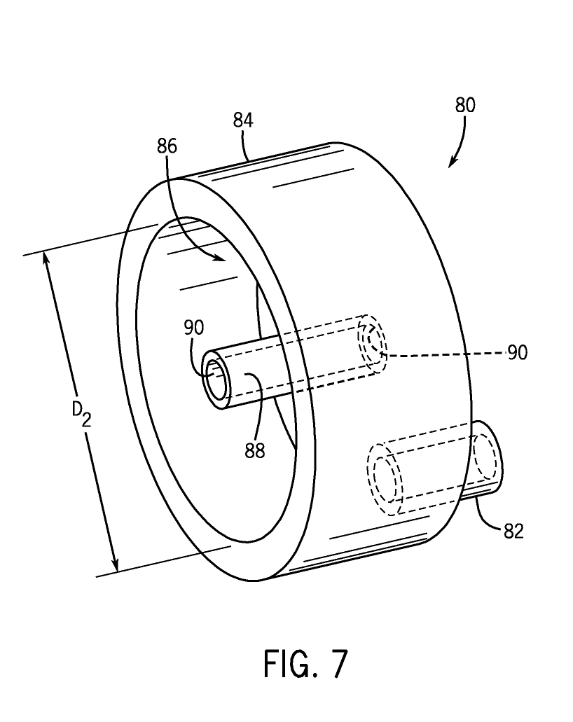

In embodiments where multiple fluid lumens 46 are used, a manifold 80 may be used to couple the multiple lumens 46 to the conduit 50 shown in FIG. 3. For example, the manifold 80 may include a connection port 82 that may couple with the conduit 50 by being inserted into the conduit 50 (or vice versa). Accordingly, secretions (e.g., blood) may be suctioned from the openings 54 and/or gases (e.g., air, oxygen) and other medicines may be delivered through the openings 54, by using the manifold 80. In one embodiment, the manifold 80 may be provided as a “cap” or circular housing, suitable for mating with the proximal base 76, as described in more detail below with respect to FIG. 7.

FIG. 7 is a perspective view of an embodiment of the manifold 80 first shown in FIG. 6. As mentioned above, the manifold 80 may be used to fluidly couple multiple lumens 56 to a single conduit 50, which may then be coupled to the medical device 48. In the depicted embodiment, the manifold 80 includes a hollow cap 84. An interior diameter (ID) of the hollow cap 84 may be equal to (or slightly less than) D2, thus enabling the insertion of the proximal base 76 of the dilator 40 inside of the hollow cap 84. In one embodiment, an interference fit (e.g., press fit, friction fit) between the proximal base 76 and the hollow cap 84 may be sufficiently strong to securely hold the components 76 and 84 in place during use. For, cleaning, the manifold 80 may be detached from the proximal base 76, for example, by using manual force. In another embodiment, a set of threads may be disposed about the proximal base 76 with matching grooves disposed inside of the hollow cap 84, and used to securely attach and detach the proximal base 76 to the manifold 80.

Gases (e.g., air, oxygen) and/or medicines may be delivered into an interior 86 of the hollow cap by using, for example, the conduit 50 attached to the connection port 82. In some embodiments, an flow blocking member 88 may be inserted inside of the inner cannula 70 and used to block flow through the inner cannula and into the hollow cap 84. The flow blocking member 88 may include a guide passage 90 suitable for inserting the guide wire 36 through the hollow cap 84, through the inner cannula 70, and into the patient. In other embodiments, the flow blocking member 88 may not be used, and the suction and/or positive pressure flow may be provided through the inner cannula 70 in addition to the flow lumens 46. By providing for the manifold 80, the multiple lumens 46 (and guide lumen 44) may be more easily connected to the medical device 48.

FIG. 8 is a rear view of the manifold 80 coupled to the proximal base 76 (shown in dashed lines) of the dilator 40. More specifically, the figure depicts a distal end of the inner cannula 70, the outer cannula 72, and the connecting members 74 used in forming the lumens 46. Also depicted are the coupling member 82 and the guide passageway 90. It is to be noted that, while in the depicted embodiment the proximal base 76 is lodged into the hollow cap 84 by using the interference fit, in other embodiments, other techniques such as threads and grooves may be used to fasten and unfasten the manifold 80 from the proximal base 76. Once attached to the proximal base 76, the manifold 80 may be coupled to the medical device 48 through the connector 82 and conduit 50, and the medical device 48 may then provide a suctioning and/or a positive pressure flow during dilation operations. In certain embodiments, as described in more detail below with respect to FIGS. 9-12, the dilator suctioning and/or positive pressure flow may enter and/or exit through the openings 54 having various shapes.

FIG. 9 is a view of the openings 54 having circular shapes 92 and 94 disposed on the outer cannula. In the depicted embodiment, the circular shapes 92 include a larger diameter as compared to the shapes 94. The smaller shapes 94 may be disposed closer to the distal tip 58, while the larger shapes 92 may be disposed further away from the distal tip 58, or vice versa. The shapes 92 and 94 may also be disposed interspersed with each other. The circular shapes 92 and 94 may be useful in providing a uniform incoming and/or outgoing flow through the openings 54. Further, because of their circular nature, the shapes 92 and 94 may present the same or similar entry/exit edge when the dilator 40 is being inserted into the trachea 12. Accordingly, dilation trauma and the entry force may be minimized.

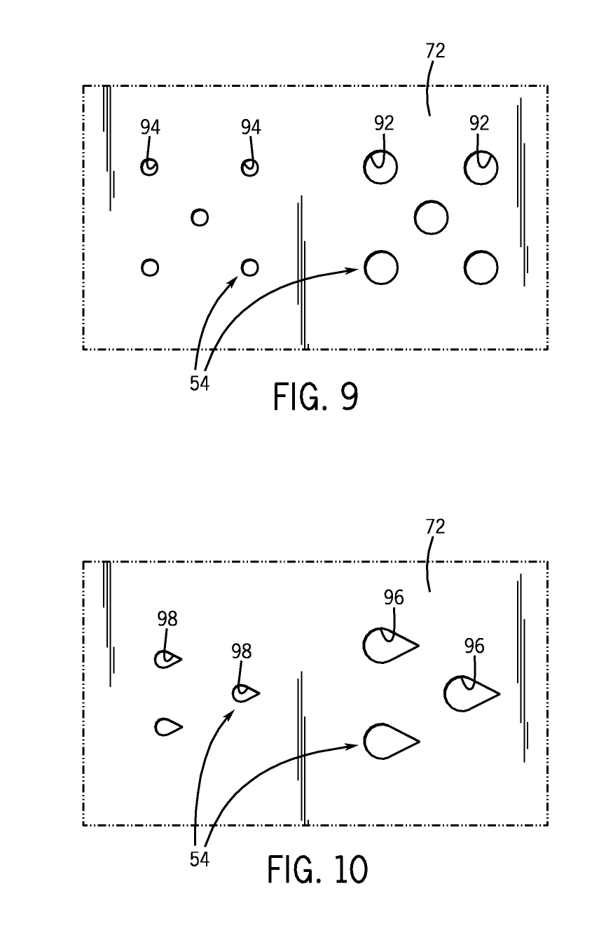

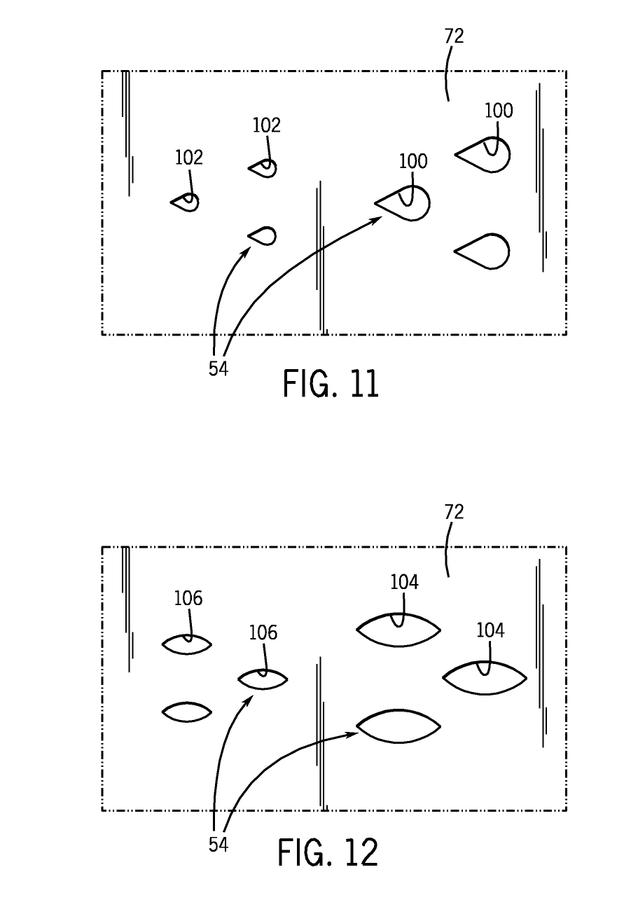

FIG. 10 is a view of the openings 54 having teardrop shapes 96 and 98. In the depicted embodiment, the teardrop shapes 96 are larger when compared to the shapes 98. The smaller shapes 98 may be disposed closer to the distal tip 58, while the larger shapes 96 may be disposed further away from the distal tip 58, or vice versa. The shapes 96 and 98 may also be disposed interspersed with each other. The teardrop shapes 96 and 98 may be useful in further minimizing dilation trauma by providing for a proximal bulb end and a distal tail end. As the outer cannula 72 moves into the trachea 12, the proximal bulb end may suction (or pressurize) the tissue first while the distal tail end may minimize trauma. If further trauma minimization is desired, the shapes 96 and/or 98 may be reversed so that the tail end enters the trachea before the bulb end during dilation, as depicted in FIG. 11.

FIG. 11 is a view of the openings 54 having reverse teardrop shapes 100 and 102. In the depicted embodiment, the reverse teardrop shapes 100 are larger when compared to the shapes 102. The smaller shapes 102 may be disposed closer to the distal tip 58, while the larger shapes 100 may be disposed further away from the distal tip 58, or vice versa. The shapes 100 and 102 may also be disposed interspersed with each other. The reverse teardrop shapes 100 and 102 may be useful in minimizing dilation trauma by providing for a proximal tail end and a distal bulb end. As the outer cannula 72 moves into the trachea 12, the proximal tail end may aid in gliding through the tissue while the distal bulb end may suction (or pressurize) the tissue. If further trauma minimization is desired, the slit-like shapes may be used, as further described in FIG. 12.

FIG. 12 is a view of the openings 54 having slit shapes 104 and 106. In the depicted embodiment, the slit shapes 104 are larger when compared to the shapes 106. The smaller shapes 106 may be disposed closer to the distal tip 58, while the larger shapes 104 may be disposed further away from the distal tip 58, or vice versa. The shapes 104 and 106 may also be disposed interspersed with each other. The slit shapes 104 and 106 may be useful in minimizing dilation trauma by providing for a proximal tail end and a distal tail end. As the outer cannula 72 moves into the trachea 12, the proximal tail end may aid in gliding through the tissue. As the outer cannula 72 is removed from the trachea 12, the distal tail end may also aid in gliding through the tissue, thus minimizing tissue trauma. It is to be noted that additional shapes may be used, including square shapes, triangle shapes, diamond shapes, oval shapes and so on, which may be easier to manufacture. Further, all of the illustrated and mentioned shapes (e.g., circles, teardrops, slits, squares, triangles, diamonds, ovals) may be provided alone or in combination with each other and fluidly coupled to the lumens 46 of the dilator 40.

What is claimed is:

1. A medical system comprising:
a tracheal dilator comprising:
a first plurality of openings;
a guide lumen configured to provide a pathway for the insertion of a guide wire during use of the tracheal dilator; and
a first flow lumen configured to fluidly couple the first plurality of openings to a medical device, wherein the medical device is configured to provide a suctioning force through the first plurality of openings during use of the tracheal dilator.

2. The system of claim 1, comprising the medical device, wherein the medical device is configured to provide a positive pressure flow of a gas during use of the tracheal dilator.

3. The system of claim 2, wherein the medical device comprises a ventilator, a pump, or a combination thereof.

4. The system of claim 1, wherein the tracheal dilator comprises an angled distal portion and a straight proximal portion, and wherein the angled distal portion comprises an angle α with the straight proximal portion.

5. The system of claim 4, wherein a comprises an angle between approximately 90° and approximately 180°.

6. The system of claim 1, wherein the tracheal dilator comprises a second plurality of openings and a second flow lumen, and wherein the second flow lumen is configured to fluidly couple the second plurality of openings to the medical device.

7. The system of claim 6, wherein the tracheal dilator comprises a manifold configured to couple the first and the second flow lumens to the medical device.

8. The system of claim 1, wherein the tracheal dilator comprises an inner cannula, an outer cannula, and a plurality of connecting members coupling the inner cannula to the outer cannula, and wherein the inner cannula comprises the guide lumen.

9. The system of claim 8, wherein a chamber formed by an outer surface of the inner cannula, an inner surface of the outer cannula, a first connecting member of the plurality of connecting members, and a second connecting member of the plurality of connecting members comprises the first flow lumen.

10. The system of claim 1, wherein at least one of the first plurality of openings comprises a circular shape, a diamond shape, a square shape, a teardrop shape, a reverse teardrop shape, a dual teardrop shape, or a combination thereof

11. A tracheal dilator comprising:
a guide cannula configured to provide a pathway for the insertion of a guide wire during use of the tracheal dilator; and
a first flow cannula comprising a first plurality of openings, wherein the first flow cannula is configured to fluidly couple the first plurality of openings to a medical device, and wherein the medical device is configured to provide a positive flow of a gas through the first plurality of openings during use of the tracheal dilator.

12. The tracheal dilator of claim 11, wherein the medical device is configured to provide a suctioning force through the first plurality of openings during use of the tracheal dilator.

13. The tracheal dilator of claim 11, comprising an angled distal portion and a straight proximal portion, and wherein the angled distal portion comprises an angle α with the straight proximal portion.

14. The tracheal dilator of claim 11, wherein a comprises an angle between approximately 90° and approximately 180°.

15. The tracheal dilator of claim 11, wherein the tracheal dilator comprises a shape increasing in diameter from a distal tip to a proximal base.

16. The tracheal tube of claim 15, wherein the shape comprises a curved shape, a conical shape, or a combination thereof.

17. The tracheal dilator of claim 11, comprising a second flow cannula comprising a second plurality of openings, wherein the second flow cannula is configured to fluidly couple the second plurality of openings to the medical device.

18. A method for manufacturing a tracheostomy dilator comprising:
manufacturing a guide cannula configured to provide a pathway for the insertion of a guide wire during use of the tracheostomy dilator; and
manufacturing a first flow cannula comprising a first plurality of openings, wherein the first flow cannula is configured to fluidly couple the first plurality of openings to a medical device, and wherein the medical device is configured to provide a positive flow of a gas through the first plurality of openings during use of the tracheal dilator.

19. The method of claim 18, wherein at least one of the plurality of openings comprises a circular shape, a diamond shape, a square shape, a teardrop shape, a reverse teardrop shape, a dual teardrop shape, or a combination thereof.

20. The method of claim 18, comprising manufacturing a second flow cannula comprising a second plurality of openings, wherein the second flow cannula is configured to fluidly couple the second plurality of openings to the medical device, and wherein the medical device is configured to provide a positive flow of the gas through the first and the second plurality of openings during use of the tracheal dilator.
